SEQUENCE LISTING (1 sequence)

SEQ ID NO: 1 - protein, 12 aa, Artificial
  RGD peptide binding ligand sequence for Integrin Receptor of HeLa cells
RGDRGDRGDPGC

(19) United States
(12) Patent Application Publication
(10) Pub. No.: US 2018/0283995 A1
(43) Pub. Date: Oct. 4, 2018

(54) METHOD AND KIT FOR MULTI-COLOR CELL IMAGING WITH DARK FIELD OPTICAL MICROSCOPY USING CONJUGATED NOBLE METAL NANOPARTICLES AS CONTRAST AGENTS

(71) Applicant: IMRA AMERICA, INC., Ann Arbor, MI (US)

(72) Inventors: Wei QIAN, Canton, MI (US); Bing LIU, Ann Arbor, MI (US)

(21) Appl. No.: 15/938,172

(22) Filed: Mar. 28, 2018

Related U.S. Application Data
(60) Provisional application No. 62/478,873, filed on Mar. 30, 2017.

(51) Int. Cl. G01N 1/30; G01N 33/58; G01N 33/574; G01N 21/552

(52) U.S. Cl. CPC G01N 1/30; G01N 33/587; G01N 2201/0633; G01N 21/554; G01N 2001/302; G01N 33/57492

(57) ABSTRACT

Disclosed is a method and a kit for multi-color cell imaging with dark field optical microscopy using noble metal conjugated nanoparticles. The noble metal conjugated nanoparticles include a stabilizer component and a binding ligand, the stabilizer component coats a portion of the noble metal nanoparticle keeping it stable in biological buffers and cell cytoplasm. The binding ligand specifically binds to targeted cells designated for imaging. The method and kit permit multicolor imaging of cells, with the multiple colors being derived from localized surface plasmon resonance of the nanoparticles, each color the result of different amounts of one or more noble metals in the nanoparticle.

### RELATED APPLICATIONS

This application claims the benefit of U.S. Provisional application Ser. No. 62/478,873 filed on Mar. 30, 2017.

### STATEMENT REGARDING FEDERALLY SPONSORED RESEARCH

NONE.

### TECHNICAL FIELD

The present disclosure relates to a method and kit for multi-color imaging of cells or tissue samples, such as a tumor cell using noble metal nanoparticle compositions and dark field optical microscopy to visualize the cells or a tissue, such as a tumor cell, with multiple colors. In certain embodiments, the present disclosure provides simple, sensitive, and inexpensive peptide-conjugated colloidal gold nanoparticles and peptide-conjugated gold-silver alloy nanoparticles which can specifically target cancer cells and enable a user to visualize cells with three different colors, green, cyan, and blue using dark field optical microscopy.

### BACKGROUND

Because of their noninvasive nature, optical imaging approaches have become indispensable tools for biomedical and biological researchers to visualize cells and intracellular biochemical and molecular processes in vitro and in vivo. Currently, the most popular optical imaging tool and method for visualizing cells, their functions and their structures is fluorescence microscopy. It is capable of tracking individually labeled molecules with high spatial and temporal resolution and of providing invaluable information regarding cells and the complex mechanisms that govern dynamical biological processes. It has thus played important roles in cell imaging and in interrogating many biological processes such as mitotic dynamics of chromosomes, centrosomes, and spindles; protein folding and migration of membrane proteins; cell signaling; and virus trafficking. Multicolor imaging can be performed using fluorescent probes with different emission wavelengths.

It is well known that fluorescence microscopy has intrinsic disadvantages and that the major drawbacks are photobleaching, photoblinking, and phototoxicity induced by the light excitation process so it is not useful for long-term (minutes to hours) cell imaging. Therefore, there is a need for developing novel imaging methods and new contrast agents with extreme photostability and biocompatibility in order to image cells and cellular processes over a long period of time without phototoxicity. Within many approaches currently being explored, imaging the scattered light from exogenous/endogenous makers is the most promising option.

Light scattering signals, which do not photobleach, quench, or decay, could overcome the identified limitations associated with using fluorescence microscopy. Noble metal nanoparticles absorb and scatter light with extraordinary efficiency. The strong interaction (absorption and scattering) of the noble metal nanoparticles with light occurs due to the enhancement by the localized surface plasmon resonance (LSPR) of nanoparticles. The physical origin of the LSPR is associated with coherent oscillations of conduction-band electrons on the noble metal nanoparticle surface upon interaction (absorption and scattering) with light. The exact LSPR band being influenced by: the size, shape, composition, and aggregation state of the nanoparticles; the dielectric properties of the surrounding medium; and by the adsorption of ions on the surface of nanoparticles. The strongly enhanced light scattering property of noble metal nanoparticles owing to the LSPR, together with their excellent photostability and biocompatibility, makes them powerful contrast agents for optical imaging with dark field illumination, which is arranged so that the light source is blocked off, causing light to scatter as it hits the specimen. Dark field illumination is ideal for viewing objects that are transparent, absorb little or no light, or have similar refractive indices as their surroundings, such as small aquatic organisms, oocytes, and cells in tissue culture.

The use of cell imaging with dark field optical microscopy based on enhanced light scattering from gold nanoparticles for the in vitro diagnosis of cancer cells via selectively targeting cancer cells with multi-functional gold nano-platforms was first reported in 2005. Since it was demonstrated in 2005, cell imaging with dark field optical microscopy using gold nanoparticles has attracted enormous attention and has become a highly used approach for in vitro cell imaging because of its advantages over the prevailing fluorescence imaging technology. For example, using an angled dark field illumination coupled with a conventional microscope, Yguerabide et al. have demonstrated that resonance light scattering from gold nanoparticles can be used as ultrasensitive labels for analyte detection in immuoassays, cells, and tissue. Yguerabide et al. “Light-scattering submicroscopic particles as highly fluorescent analogs and their use as tracer labels in clinical and biological applications”, Analytical Biochemistry, 1998 Sep. 10; 262(2): 157-176. In addition, the studies by Sokolov et al. showed that gold nanoparticle-labeled cervical cancer cells and tissues can be well resolved from normal ones upon illumination with single wavelength laser light either from a simple laser pen or the excitation laser light from a confocal microscope. Sokolov et al. “Real-time vital optical imaging of precancer using anti-epidermal growth factor receptor antibodies conjugated to gold nanoparticles”, Cancer Research 2003 May 1; 63(9): 1999-2004. Furthermore, several groups have extended the applicability of cell imaging with dark field optical microscopy to monitor the dynamic interactions between biomolecules in live cells in real time. See Aaron et al. “Dynamic Imaging of Molecular Assemblies in Live Cells Based on Nanoparticle Plasmon Resonance Coupling”, Nano Lett. 2009, 9 (10), 3612-3618 and Rong et al. “Resolving Sub-Diffraction Limit Encounters in Nanoparticle Tracking Using Live Cell Plasmon Coupling Microscopy”, Nano Lett. 2008, 8 (10), 3386-3393.

Although significant progress has been made in cell imaging with dark field optical microscopy using noble metal nanoparticles, the development of this technology is still in its infancy. For example, there is no demonstration so far of using this technology for the distinct multicolor cell imaging, which is necessary for the observation of the spatial relationship and temporal dynamics of subcellular constituents and the simultaneous detection of different cell surface receptors. Therefore, there is an urgent demand for simple, low-cost, highly sensitive methods capable of providing cell imaging with multiple colors using dark field optical microscopy.

### SUMMARY OF THE DISCLOSURE

The present disclosure relates to methods and kits for the multi-color cell imaging of target cells or tissues with dark field optical microscopy using noble metal nanoparticle bioconjugates. The noble metal nanoparticle-biomolecule conjugates are designed to have both a covalently bound stabilizer component and a covalently bound binding ligand conjugated onto their surface so that they can target cells of interest and also they are stable in the biologically relevant environment, such as a cell culture medium or the cytoplasm of a cell. The imaging protocols are designed to prevent aggregation of the noble metal nanoparticle-biomolecule conjugates so that they maintain their intrinsic non-aggregated localized surface plasmon resonance (LSRP) peak.

In some embodiments, the present disclosure provides methods for a green color cell imaging by dark field optical microscopy. In this embodiment, the noble metal nanocolloids used for cell imaging are 30 nm peptide-conjugated pure gold nanocolloids that have been conjugated with a stabilizer component and a binding ligand. In a first step the 30 nm peptide-conjugated pure gold nanocolloids are added to a cell culture dish containing the cells designated for imaging and the cells are incubated in the presence of the peptide-conjugated nanoparticles for less than 2 hours at 37° C. and 5% CO₂ in a humidified incubator to allow for cell labeling with these peptide-conjugated nanoparticles. In a second step the medium in the cell culture dish is gently aspirated at the end of incubation period and then, the cells are washed with a balanced salt solution rinse buffer of Dulbecco's Phosphate Buffered Saline (DPBS) three times to remove free peptide-conjugated gold nanoparticles before imaging. In a third step DPBS is added to the cell culture dish and then the cells are ready for the optical microscopy imaging using dark field illumination with a lamp (halogen or xenon) or light-emitting diode. In a fourth step cell imaging is performed on the cells stained with the 30 nm peptide-conjugated gold nanoparticles in the cell culture dish. An inverted optical microscope is preferred for imaging from below the cell culture dish. Limited by the working distance, up to 50× objective lens can be used. Under dark field illumination, cells stained with the peptide-conjugated gold nanoparticles mostly appear green, which is attributed to enhanced light scattering by the gold nanoparticles. Yellow and orange colors occasionally appear at high concentrations due to the formation of peptide-conjugated gold nanoparticle aggregates occurring in the cytoplasm.

In some embodiments, the present disclosure provides methods for cyan colored cell imaging by dark field optical microscopy. In this embodiment, the noble metal nanocolloids used for cell imaging are 30 nm peptide-conjugated gold-silver alloy nanoparticles with 80% gold mole fraction and 20% silver mole fraction, therefore denoted as Au80Ag20. In a first step the 30 nm peptide-conjugated Au80Ag20 alloy nanoparticles are added to the cell culture dish containing the cells designated for imaging and the cells are incubated in the presence of the conjugated nanoparticles for less than 2 hours at 37° C. and 5% CO₂ in a humidified incubator to allow for cell labeling with these conjugated nanoparticles. In a second step the medium in the cell culture dish is gently aspirated at the end of incubation and then, the cells are washed with Dulbecco's Phosphate Buffered Saline (DPBS) three times to remove free peptide-conjugated Au80Ag20 alloy nanoparticles before imaging. In a third step DPBS buffer is added to the cell culture dish and the cells are ready for the optical microscopy imaging using dark field illumination with a lamp (halogen or xenon) or light-emitting diode. In a fourth step cell imaging is performed for cells stained with the 30 nm peptide-conjugated Au80Ag20 alloy nanoparticles in the cell culture dish. An inverted optical microscope is preferred for imaging from below the cell culture dish. Limited by the working distance, up to 50× objective lens can be used. Under dark field illumination, cells stained with peptide-conjugated Au80Ag20 alloy nanoparticles mostly appear cyan, which is attributed to enhanced light scattering by the Au80Ag20 alloy nanoparticles. Green and yellow colors occasionally appear at high concentrations due to the formation of Au80Ag20 alloy nanoparticle aggregates occurring in the cytoplasm.

In some embodiments, the present disclosure provides methods for a blue color cell imaging by dark field optical microscopy. In this embodiment, the noble metal nanocolloids used for cell imaging are 30 nm peptide-conjugated gold-silver alloy nanoparticles with 50% gold mole fraction and 50% silver mole fraction (therefore denoted as Au50Ag50). In a first step 30 nm peptide-conjugated Au50Ag50 alloy nanoparticles are added to the cell culture dish containing the cells designated for imaging and the cells are incubated in the presence of the conjugated nanoparticles for less than 2 hours at 37° C. and 5% CO₂ in a humidified incubator to allow for cell labeling with these conjugated nanoparticles. In a second step the medium in the cell culture dish is gently aspirated at the end of incubation and then, the cells are washed with Dulbecco's Phosphate Buffered Saline (DPBS) three times to remove free peptide-conjugated Au50Ag50 alloy nanoparticles in solution before imaging. In a third step DPBS buffer is added to the cell culture dish and the cells are ready for the optical microscopy imaging using dark field illumination with a lamp (halogen or xenon) or light-emitting diode. In a fourth step cell imaging is performed for cells stained with the 30 nm Au50Ag50 alloy conjugated nanoparticles in the cell culture dish. An inverted optical microscope is preferred for imaging from below the cell culture dish. Limited by the working distance, up to 50× objective lens can be used. Under dark field illumination, cells stained with the peptide-conjugated Au50Ag50 alloy nanoparticles mostly appear blue, which is attributed to enhanced light scattering by the Au50Ag50 alloy nanoparticles. Cyan and green colors occasionally appear at high concentrations due to the formation of Au50Ag50 alloy nanoparticle aggregates occurring in the cytoplasm.

### BRIEF DESCRIPTION OF THE DRAWINGS

FIG. 1A is a schematic illustration of a process according to the present disclosure of producing peptide-conjugated nanoparticles;

FIG. 1B shows the increase in diameter as measured by dynamic light scattering of bare 30 nm gold nanoparticles that have been conjugated with the illustrated ratios of thiolated polyethylene glycol having a molecular weight of 5,000;

FIG. 1C shows hydrodynamic diameters of partially PEGylated colloidal gold nanoparticles conjugated with different amounts of RGD peptide with the amino acid sequence of SEQ. NO. 1 RGDRGDRGDPGC;

FIG. 2 schematically illustrates a laser-based ablation system for the top-down production of colloidal noble metal nanoparticles in a liquid in accordance with the present disclosure;

FIG. 3A illustrates the UV-VIS absorption spectra of various stable colloidal noble metal nanoparticles, including Au20Ag80 alloy nanoparticles (solid line), Au50Ag50 alloy nanoparticles (dashed line), Au80Ag20 alloy nanoparticles (dotted line), pure Au nanoparticles (dash-dot line), and Au50Cu50 alloy nanoparticles (dash-dot-dot line) prepared according to the present disclosure;

FIG. 3B shows a transmission electron microscopy (TEM) picture of stable colloidal gold nanoparticles with an average particle diameter of 30 nanometers;

FIG. 4A illustrates dark field illumination and FIG. 4B illustrates bright field illumination;

FIG. 5A displays an image of HeLa cancer cells with dark field optical microscopy stained using 30 nm colloidal peptide-conjugated gold nanoparticles prepared according to the present disclosure as contrast agents;

FIG. 5B displays a transmission electron microscopy (TEM) micrograph of intercellular 30 nm peptide-conjugated gold nanoparticles (black dots) which shows that the gold nanoparticles exist and aggregate in the cytoplasm;

FIG. 6 illustrates that under dark field illumination, there are changes in the color of cells stained with 30 nm peptide-conjugated gold nanoparticles depending on the incubation time due to the formation of aggregates of 30 nm peptide-conjugated gold nanoparticles in the cytoplasm over long periods of incubation time;

FIG. 7 illustrates that under dark field illumination, there are changes in the color of cells stained with 30 nm peptide-conjugated Au50Ag50 alloy nanoparticles depending on the incubation time due to the formation of aggregates of 30 nm peptide-conjugated Au50Ag50 alloy nanoparticles in the cytoplasm over long periods of incubation; and

FIG. 8 displays multi-color cell imaging with dark field optical microscopy using 30 nm peptide-conjugated gold nanoparticles (left), 30 nm peptide-conjugated Au80Ag20 alloy nanoparticles (middle), and 30 nm peptide-conjugated Au50Ag50 alloy nanoparticles (right) as contrast agents.

### DETAILED DESCRIPTION

We disclose in the present disclosure a novel method and kit containing biomolecule-conjugated colloidal noble metal nanoparticles designed for dark field optical microscopy imaging of cells with multiple colors. The colloidal noble metal nanoparticles are conjugated with both a stabilizer component and a binding ligand. The stabilizer component covers from 30 to 70% of the available surface area of the nanoparticles and the binding ligand covers the rest of the nanoparticle surface. The binding ligand is selected to allow the conjugated nanoparticles to target specific cells or tissues of interest and gives the conjugated nanoparticle its specificity. Throughout the specification and examples Dulbecco's Phosphate Buffered Saline (DPBS) is used as the balanced salt solution in treating cells during various processes, as discussed herein and know to one of skill in the art other balanced salt solutions could be used. The purpose of the balanced salt solution is to maintain the cells without causing shrinkage or swelling with possible bursting due to changes in osmolality of the solution that the cells are in.

Throughout the present specification and claims the terms “conjugated nanoparticle”, “peptide-conjugated nanoparticles”, “noble metal nanoparticle-biomolecule conjugates” and “noble metal nanoparticle conjugates” are used interchangeably and are meant to refer to a colloidal noble metal nanoparticle that has been conjugated with a stabilizer component and a binding ligand. The stabilizer component covers from 30 to 70% of the surface of the nanoparticle and the binding ligand covers the rest of the nanoparticle surface. Suitable stabilizer components are discussed herein. As discussed herein the binding ligand conveys the specificity to the conjugated nanoparticle and allows it to target specific cell types or tissue types.

The noble metals finding special use in the present disclosure preferably comprise: gold, silver, copper, palladium, platinum, and alloys comprising one or more of these noble metals in any combination. The specific examples within the disclosure are not meant to be limiting, but illustrative of the disclosure.

Under dark field illumination, the noble metal nanoparticles which are conjugated with binding ligands for specific cell targeting, the conjugated nanoparticles of the present disclosure, are 10⁵ to 10⁶ times brighter than organic dyes due to the particles' large optical scattering cross-section at the plasmon resonance wavelength. Therefore, low concentrations of noble metal nanoparticle conjugates on the order of sub nanomolar concentrations are sufficient to produce a sharp image. Sharp images can be produced using a concentration of the conjugated nanoparticle in the range of 300 picomolar or less. In addition, noble metal nanoparticle conjugates are also resistant to photo-blinking and photo-bleaching, allowing continuous and extended cell imaging, tracking, and analysis.

Colloidal noble metal nanoparticles are metal nanoparticles dispersed in a dispersion medium, typically water, but other media can also be used as discussed below. Noble metal nanoparticles have attracted substantial interest from scientists for over a century because of their unique physical, chemical, and surface properties, such as: (i) size, shape, and composition-dependent strong optical extinction and scattering which is tunable from ultraviolet (UV) wavelengths all the way to near infrared (NIR) wavelengths; (ii) large surface areas for conjugation to functional ligands; and (iii) little or no long-term toxicity or other adverse effects in vivo allowing their high acceptance level in living systems.

Currently, the overwhelming majority of noble metal nanocolloids are prepared by using the standard wet chemical methodology. For example, gold nanocolloids are prepared by sodium citrate reduction of chloroauric acid (HAuCl₄) and gold-silver alloy nanocolloids with varying mole fractions of gold and silver within the same individual nanoparticles, which results in tunable localized surface plasmon resonance between 400 nm and 540 nm, are prepared by co-reduction of chlorauric acid (HAuCl₄) and silver nitrate (AgNO₃) with sodium citrate. The mole fraction of gold of gold-silver alloy nanoparticle means the fraction of total atoms within gold-silver alloy nanoparticle that are gold and the mole fraction of silver of gold-silver alloy nanoparticle means the fraction of total atoms within gold-silver alloy nanoparticle that are silver. This method results in the synthesis of spherical noble metal nanoparticles with diameters ranging from 5 to 200 nanometers (nm) which are capped or covered with negatively charged citrate ions. The citrate ion capping prevents the nanoparticles from aggregating by providing electrostatic repulsion between nanoparticles.

Other wet chemical methods for formation of colloidal gold nanoparticles include the Brust method, the Perrault method and the Martin method. The Brust method relies on reaction of chlorauric acid with tetraoctylammonium bromide in toluene and sodium borohydride. The Perrault method uses hydroquinone to reduce the HAuCl₄ in a solution containing gold nanoparticle seeds. The Martin method uses reduction of HAuCl₄ in water by NaBH₄ wherein the stabilizing agents HCl and NaOH are present in a precise ratio. All of the wet chemical methods rely on first converting gold (Au) with a strong acid into the atomic formula HAuCl₄ and then using this atomic form to build up the nanoparticles in a bottom-up type of process. All of the methods require the presence of stabilizing agents to prevent the gold nanoparticles from aggregating and precipitating out of solution.

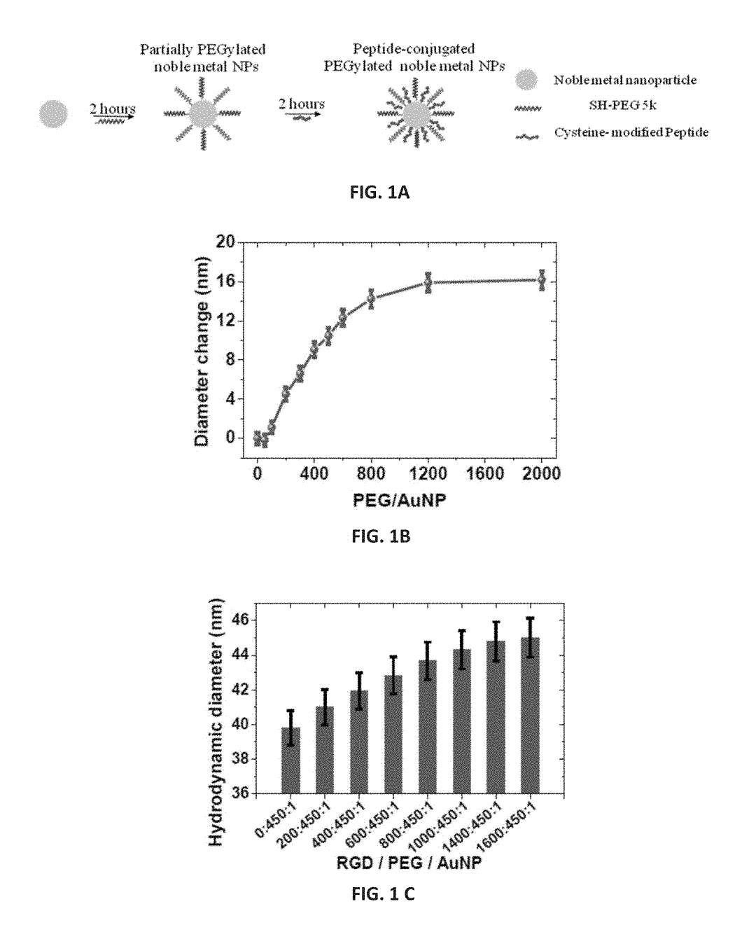

In addition to the wet chemical methods, several physical methods exist for making noble metal nanoparticles. One of these physical methods of making noble metal nanoparticles is based on pulsed laser ablation of a noble metal target immersed in a liquid, and it has been attracting increasingly widespread interest. In contrast to the chemical procedures, pulsed laser ablation of a noble metal target immersed in a liquid offers the possibility of generating reactant/surfactant-free and chemically pure stable noble metal nanocolloids, meaning bare nanoparticles that have no surface modifications, which allows for sequential conjugation of both the stabilizer component, for example, thiolated PEG molecules with molecular weight of 5000, SH-mPEG 5k as used in the present disclosure and the binding ligand, for example, RGD peptides with an amino acid sequence of RGDRGDRGDPGC SEQ. NO. 1, used in the present disclosure onto their surface. As demonstrated in FIG. 1A, the method of “sequential conjugation” offers the capability of precisely tuning the ratios of two types of ligands, the stabilizer component and the binding ligand, bound to noble metal nanoparticles for the optimization of stability, biocompatibility, and targeting ability of the obtained noble metal conjugated nanoparticle.

In the present specification and claims, sequences of amino acids of any peptides are written via the convention that the left end of the sequence is the amino terminal end while the right end is the carboxyl terminal end and either the accepted three letter abbreviations for each amino acid or their single letter abbreviations will be used.

As discussed above, the overwhelming majority of commercially available noble metal nanoparticles are prepared by the standard sodium citrate reduction reaction. This method is a bottom-up method and allows for the synthesis of spherical noble metal nanoparticles with diameters ranging from 1 to 200 nanometers (nm) which are capped with negatively charged citrate ions. The capping controls the growth of the nanoparticles in terms of rate, final size, geometric shape and electrostatic repulsion stabilizes the nanoparticles against aggregation.

In contrast to the prior process of bottom-up fabrication using wet chemical processes, the noble metal nanocolloids used in the present disclosure are produced by a top-down nanofabrication approach. The top-down fabrication methods of the present disclosure start with a bulk material in a liquid and then break the bulk material into nanoparticles in the liquid by applying physical energy to the material. The physical energy can be mechanical energy, heat energy, electric field arc discharge energy, magnetic field energy, ion beam energy, electron beam energy, or laser beam energy including laser ablation of the bulk material. The present process produces reactant/surfactant-free and chemically pure colloidal noble metal bare nanoparticles that are stable in the ablation liquid and avoids the wet chemical issues of residual chemical precursors, stabilizing agents and reducing agents.

The term “stable” as applied to a colloidal noble metal preparation prepared according to the present disclosure refers to stability of the absorbance intensity caused by localized surface plasmon resonance (LSPR) of a bare colloidal noble metal preparation upon storage. Generally, if a colloidal noble metal preparation becomes unstable the noble metal nanoparticles begin to aggregate and precipitate out of the suspension over time, thus leading to a decrease in the absorbance at the localized surface plasmon resonance. A “stable” colloidal noble metal preparation is one that exhibit less than a 10% decrease in the absorbance at the peak LSPR for the preparation over the measured time interval. Thus, a preparation is “stable” for 2 weeks, for example, if the absorbance measured a time 0 and after 2 weeks is within 10% at the peak LSPR. In addition, “stable” means that there is a minimal red shift or change in localized surface plasmon resonance of 2 nanometers or less over the storage time.

The top-down nanofabrication approaches according to the present disclosure all require that the generation of the nanoparticles from the bulk material occur in the presence of a suspension medium. In one embodiment, the process comprises a one step process wherein the application of the physical energy source, such as mechanical energy, heat energy, electric field arc discharge energy, magnetic field energy, ion beam energy, electron beam energy, or laser energy to the bulk gold occurs in the suspension medium. The bulk source is placed in the suspension medium and the physical energy is applied thus generating nanoparticles that are immediately suspended in the suspension medium as they are formed. In another embodiment, the present disclosure is a two-step process including the steps of: 1) fabricating noble metal nanoparticle arrays on a substrate by using photo, electron beam, focused ion beam, nanoimprint, or nanosphere lithography as known in the art; and 2) removing the noble metal nanoparticle arrays from the substrate into the suspension liquid using one of the above described physical energy methods. In both the one step and two step methods the noble metal nanocolloid is formed in situ by generating the nanoparticles in the suspension medium using one of the physical energy methods.

Among the unique optical and electronic properties of noble metal nanocolloids mentioned above, their localized surface plasmon resonance (LSPR) has received particular interest. The physical origin of the LSPR is associated with coherent oscillations of conduction-band electrons on the noble metal nanoparticle surface upon interaction, absorption and scattering, with light with the exact LSPR band being sensitive to: the size, shape, composition, and aggregation state of the nanoparticles; to the dielectric properties of the surrounding medium; and to the adsorption of ions on the surface of nanoparticles. For example, for gold (Au) nanocolloids with an average particle diameter of 30 nm, the maximum absorbance of the localized surface plasmon resonance of this Au nanocolloid is at 530 nm, this is the peak LSPR. Because the LSPR of 30 nm gold nanocolloids is around 530 nm, the 30 nm gold nanocolloids scatter strongly a green color, which will make the gold nanocolloid show as a green color under dark field microscopy. For 30 nm gold-silver alloy nanoparticles with 80% gold mole fraction and 20% silver mole fraction, therefore denoted as Au80Ag20, the maximum absorbance of the localized surface plasmon resonance of this Au80Ag20 nanocolloid is at 500 nm. Therefore, the 30 nm Au80Ag20 nanocolloids scatter strongly a cyan color, which will make Au80Ag20 nanocolloids show as a cyan color under dark field microscopy. For 30 nm gold-silver alloy nanoparticles with 50% gold mole fraction and 50% silver mole fraction, therefore denoted as Au50Ag50, the maximum absorbance of the localized surface plasmon resonance of this Au50Ag50 nanocolloid is at 450 nm. Therefore, the 30 nm Au50Ag50 nanocolloids scatter strongly a blue color, which will make Au80Ag20 nanocolloids show as blue in color under dark field microscopy. The property of sensitive dependence of LSPR on the composition of noble metal nanocolloid, which results in tunable LSPR throughout the visible region of the electromagnetic spectrum, enables one following the present disclosure to design a novel method for multi-color cell imaging with dark-field optical microscopy using noble metal nanocolloids as contrast agents.

Taking advantage of this property, in the present disclosure, we have developed methods and kits for multi-color cell imaging with dark field optical microscopy using noble metal nanocolloids as contrast agents. As noble metal nanocolloids have extremely high extinction coefficients, which are more than 10⁵ to 10⁶ times higher than those of organic dyes, a low concentration of noble metal nanocolloids on the order of sub-nanomolar is sufficient to produce a cell image of sharp contrast compared to the concentration of micromolar in the case of using conventional fluorescent dyes and proteins. In addition, neither excitation nor emission filters are required for obtaining multi-color cell imaging using the strongly enhanced light scattering signals from noble metal nanocolloids designed according to the present disclosure.

Therefore, noble metal nanocolloid-based kits developed in this disclosure for multi-color cell imaging with dark field optical microscopy will provide the following advantages: (1) the concentration of noble metal nanocolloid reagents required for cell imaging is low; (2) a cell staining procedure with noble metal nanocolloid reagents is simple and the time required for cell staining is short, less than 2 hours; (3) removing the noble metal nanocolloid reagent solution or washing the cells is not necessary; 4) the noble metal nanocolloid reagent is retained by the stained cells after fixation allowing fixed cell imaging; and (5) multicolor imaging can be obtained with simple and low-cost imaging platform.

In at least one embodiment of the present disclosure, noble metal nanocolloids, such as Au nanocolloid, Au80Ag20 alloy nanocolloid, Au50Ag50 alloy nanocolloid, Au20Ag80 alloy nanocolloid, and Au50Cu50 alloy nanocolloid, were produced by pulsed laser ablation of a bulk noble metal target, such as pure Au, Au80Ag20, Au50Ag50 alloy, Au20Ag80 alloy, and Au50Cu50 alloy in deionized water as the suspension medium. The specific ratios refer to the molar fraction % of the elements in the alloy, thus Au20Ag80 has 20% mole fraction of Au and 80% mole fraction of Ag.

The process of the present disclosure for producing conjugated nanoparticles by the sequential conjugation is shown in FIG. 1A for both conjugation steps. As shown in FIG. 1A both the polyethylene glycol (PEG) and the peptide are conjugated onto the noble metal nanoparticle for the fabrication of the conjugated nanoparticle which can be used as a contrast agent for specific targeting of cells of interest and for imaging with dark field optical microscopy. Conjugation is performed by first adding thiolated PEG molecules, in one embodiment with a molecular weight of 5000 (SH-mPEG 5k). The SH-mPEG is reacted in an amount high enough to permit stability of the nanoparticles in both cell culture medium and the cell cytoplasm while still less than the amount that provides for 100% surface coverage such that there are unoccupied sites on the surface of the noble metal nanoparticle for subsequent direct conjugation of the binding ligand, a peptide in this example, onto the surface of the noble metal nanoparticles. Both the stabilizing component and the binding ligand are directly bonded to the surface of the nanoparticle via covalent linkages. In one example the PEG is bonded to the surface via a thiol bond. In another example the binding ligand is a peptide sequence that targets a cell surface receptor and it is bonded to the nanoparticle via a thiol bond provided by the amino acid cysteine. None of the conjugation bonds in the present disclosure require any integrating molecules or other linker molecules, both the stabilizer and the binding ligand are directly bonded to the nanoparticle surface. The method of “Sequential Conjugation” enables the precise control of the ratio of the two types of ligands bound to the noble metal nanoparticles for optimization of stability, biocompatibility, and targeting ability of the obtained noble metal conjugated nanoparticles. In FIG. 1B one sees displayed the diameter change of 30 nm colloidal gold nanoparticles after being PEGylated, the process of conjugating PEG to the nanoparticle, with different amounts of SH-PEG 5k molecules, the first step of the sequential conjugation, measured by dynamic light scattering (DLS). The X-axis “PEG/AuNP” represents the molar ratio of SH-PEG 5k molecules to colloidal gold nanoparticles. One sees the size change is maximal at about 1200 PEG/AuNP, this represents saturation of the surface with PEG for this size nanoparticle. This process can be used with any size gold nanoparticle to determine the 100% coverage level by the selected stabilizing component, in this case PEG. In all of the experimental data presented in the present specification, when binding PEG to 30 nm pure Au nanoparticles in the first step of formation of the conjugated nanoparticles the PEG/AuNP ratio used was 450:1 which as can be seen from FIG. 1B provides for partial coverage leaving free space for binding of the binding ligand in the second step. In the second step the nanoparticles are conjugated to the binding ligand. As discussed herein the binding ligand can be any of a variety of ligands that are capable of covalently binding to the nanoparticles and of targeting a cell or tissue of interest. For illustrative purposes in the present specification the selected binding ligand was a form of RGD peptide which is known to target the integrin receptor found on HeLa cells. The specific binding ligand selected had the amino acid sequence SEQ. NO. 1 RGDRGDRGDPGC. In FIG. 1C the increase in hydrodynamic size is plotted against increasing amounts of RGD peptide added to the nanoparticles that had gone through step 1 and had been PEGylated. The C in the RGD sequence, SEQ. NO. 1, provides a thiol bond that binds to the nanoparticles. The results show the size of the gold nanoparticles increases along with the increase of the molar ratios of RGD peptide molecules to colloidal gold nanoparticles (RGD/AuNP).

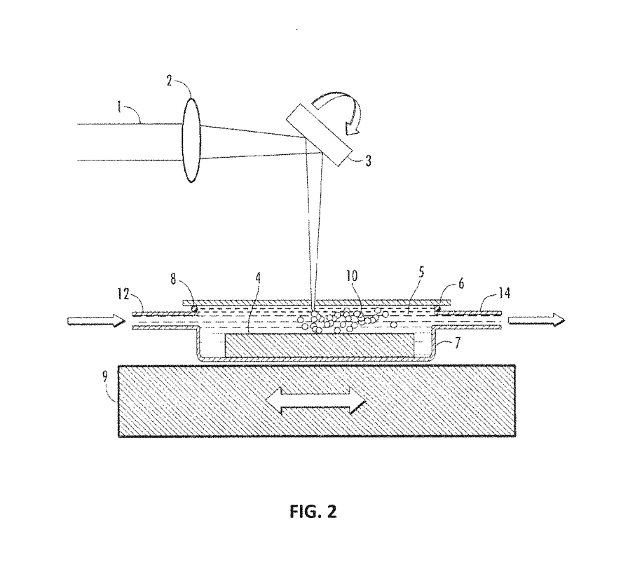

FIG. 2 schematically illustrates a laser-based system for producing colloidal suspensions of noble metal nanoparticles of complex compounds such as gold in a liquid using pulsed laser ablation in accordance with the present disclosure. The nanoparticles produced according to this system are bare, meaning they have no surface modifications, and they are used as the source material for the process as shown in FIG. 1A. In one embodiment a laser beam 1 is generated from an ultrafast femtosecond pulsed laser source, not shown, and focused by a lens 2. The source of the laser beam 1 can be a pulsed laser or any other laser source providing suitable pulse duration, repetition rate, and/or power level as discussed below. The focused laser beam 1 then passes from the lens 2 to a guide mechanism 3 for directing the laser beam 1 at a target 4 of the bulk material. Alternatively, the lens 2 can be placed between the guide mechanism 3 and a target 4 of the bulk material. The guide mechanism 3 can be any of those known in the art including piezo-mirrors, acousto-optic deflectors, rotating polygons, a vibration mirror, or prisms. Preferably the guide mechanism 3 is a vibration mirror 3 to enable controlled and rapid movement of the laser beam 1. The guide mechanism 3 directs the laser beam 1 to a target 4. In one embodiment, the target 4 is a bulk gold target. The target 4 is submerged a distance, from several millimeters to preferably less than 1 centimeter, below the surface of a suspension liquid 5. The target 4 is positioned in a container 7 additionally but not necessarily having a removable glass window 6 on its top. Optionally, an O-ring type seal 8 is placed between the glass window 6 and the top of the container 7 to prevent the liquid 5 from leaking out of the container 7. Additionally but not necessarily, the container 7 includes an inlet 12 and an outlet 14 so the liquid 5 can be passed over the target 4 and thus be re-circulated. The container 7 is optionally placed on a motion stage 9 that can produce translational motion of the container 7 with the target 4 and the liquid 5 relative to the laser beam 1. Flow of the liquid 5 is used to carry the nanoparticles 10 generated from the target 4 out of the container 7 to be collected as a colloidal suspension. The flow of liquid 5 over the target 4 also cools the laser focal volume. The liquid 5 can be any liquid that is largely transparent to the wavelength of the laser beam 1, and that serves as a colloidal suspension medium for the target material 4. In one embodiment, the liquid 5 is deionized water having a resistivity of greater than 0.05 MOhm·cm, and preferably greater than 1 MOhm·cm. In other embodiments the liquid 5 can comprise other suspension liquids including, for example, a physiological buffer solution, a phosphate buffered saline or other suitable media. The system thus allows for generation of colloidal gold nanoparticles in situ in a suspension liquid so that a colloidal gold nanoparticle suspension is formed. The formed gold nanoparticles are immediately and stably suspended in the liquid and thus no dispersants, stabilizer agents, surfactants or other materials are required to maintain the colloidal suspension in a stable state. This result allows the creation of a unique colloidal gold suspension that contains bare gold nanoparticles.

The following laser parameters were used to fabricate noble metal nanocolloids by pulsed laser ablation of a bulk noble metal target in deionized water: a pulse energy of 10 micro Joules (μJ), a pulse repetition rate of 100 kHz, a pulse duration of 700 femtoseconds (fs), and a laser spot size on the ablation target of about 50 microns (O. For the preparation of noble metal nanocolloids according to the present disclosure, a 16 millimeter (mm) long, 8 mm wide, and 0.5 mm thick rectangular target of noble metal from Alfa Aesar was used. For convenience, the noble metal target materials can be attached to a bigger piece of a bulk material such as a glass slide, another metal substrate, or a Si substrate.

More generally, for the fabrication of noble metal nanocolloids used in the present disclosure, the suitable laser ablation parameters are as follows: a pulse duration in a range of from about 10 fs to about 500 picoseconds (ps), preferably from about 100 fs to about 30 ps; a pulse energy in the range of from about 1 μJ to about 100 μJ; a pulse repetition rate in the range of from about 10 kHz to about 10 MHz; and the laser spot size may be less than about 100 μm. The target material has a size in at least one dimension that is greater than a spot size of a laser spot at a surface of the target material.

Samples of colloidal noble metal nanoparticles prepared by laser ablation in deionized water were characterized by commercially available analytic instruments and techniques, including UV-VIS absorption spectra and transmission electron microscopy (TEM). UV-VIS absorption spectra were recorded with a Shimadzu UV-3600 UV-VIS-NIR spectrophotometer. Noble metal nanoparticles were visualized using transmission electron microscopy (TEM; JEOL 2010F, Japan) at an accelerating voltage of 100 kilovolts (kV). All measurements and processes were carried out at room temperature, approximately 25° C.

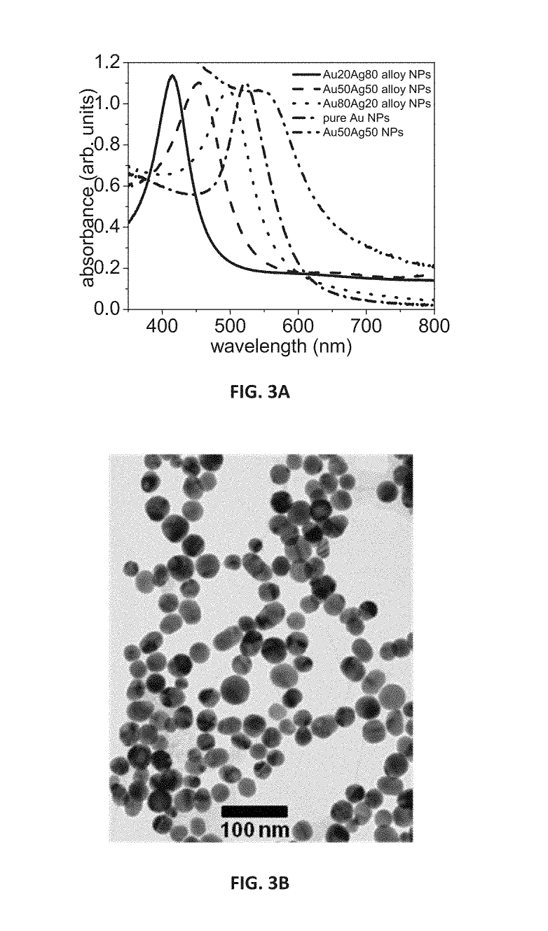

FIG. 3A shows the UV-VIS absorption spectra of noble metal nanoparticles of various stable colloidal noble metal nanoparticles, including Au20Ag80 alloy nanoparticles (solid line), Au50Ag50 alloy nanoparticles (dashed line), Au80Ag20 alloy nanoparticles (dotted line), pure Au nanoparticles (dash-dot line), and Au50Cu50 alloy nanoparticles (dash-dot-dot line) prepared according to the present disclosure by laser ablation of various corresponding noble metal targets in deionized water, which shows tunable LSPR throughout the visible region of the electromagnetic spectrum via varying the composition of the noble metal nanoparticles. The LSPR of Au20Ag80 alloy nanoparticles is around 410 nm, the LSPR of Au50Ag50 alloy nanoparticles is around 450 nm, the LSPR of Au80Ag20 alloy nanoparticles is around 490 nm, the LSPR of Au nanoparticles is around 530 nm, and the LSPR of Au50Cu50 alloy nanoparticles is around 560 nm. FIG. 3B shows a Transmission Electron Microscopy (TEM) picture of a preparation of stable bare colloidal gold nanoparticles with an average particle diameter of 30 nanometers prepared by laser ablation in deionized water according to the present disclosure. In the present specification all of the cell experiments involving noble metal nanocolloids were conducted using noble metal nanocolloids with the final optical density in the cell culture medium being OD=1 as measured at the peak LSPR for the preparation.

After the fabrication of the noble metal nanocolloids, both the stabilizer component and the binding ligand are conjugated onto their surface according to the method of “Sequential Conjugation” demonstrated in FIG. 1A. In one embodiment as described, the stabilizer component is chosen to be a thiolated polyethylene glycol (PEG) molecule with molecular weight of 5000 (SH-mPEG 5k) because it is a biocompatible material that can improve colloidal stability of noble metal nanoparticles in biological media of high ionic strength, meaning for example greater than or equal to 50 mM NaCl, and to minimize nonspecific interactions of noble metal nanoparticles with biomolecules, cells, and tissues. In one embodiment the binding ligand is chosen to be a peptide because of the following advantages, including (1) inexpensive to produce; (2) high specificity and binding activity; (3) great stability and low toxicity; and (4) high organ and tumor penetration. In one embodiment, both SH-PEG 5k molecules and RGD peptides with the amino acid sequence being SEQ. NO. 1 RGDRGDRGDPGC were conjugated onto the surface of 30 nm colloidal gold nanoparticles using the method of “Sequential Conjugation” as shown in FIG. 1A.

The PEG is a linear polymer having of repeated units of —CH₂—CH₂—O—. Depending on the molecular weight, the same molecular structure is also termed poly (ethylene oxide) or polyoxyethylene. The polymer is very soluble in a number of organic solvents as well as in water. After being conjugated onto the surfaces of noble metal nanoparticles, in order to maximize entropy, the PEG chains have a high tendency to fold into coils or bend into a mushroom like configuration with diameters much larger than proteins of the corresponding molecular weight. The surface modification of noble metal nanoparticles with PEG is often referred to as ‘PEGylation’ and in the present specification and claims binding of PEG to noble metal nanoparticles will be referred to as PEGylation. Since the layer of PEG on the surface of noble metal nanoparticles can help to stabilize the noble metal nanoparticles in an aqueous environment by providing a steric barrier between interacting noble metal nanoparticles, PEGylated noble metal nanoparticles are much more stable in biological buffers and the cellular cytoplasm.

In addition, all kinds of PEG molecules, comprising mono-, homo-, and heterofunctional PEG with different functional groups and one or multiple arms and molecular weights ranging from 200 Da to 100,000,000 Da can also be used as the stabilizer component. The PEG used as a stabilizer component can be a thiolated PEG having a molecular weight of from 200 Daltons to 100,000,000 Daltons. It can be a mono- homo or hetero-functional PEG having branches. Examples of polymers other than PEG that can be used as stabilizer components include polyacrylamide, polydecylmethacrylate, polymethacrylate, polystyrene, dendrimer molecules, polycaprolactone (PCL), polylactic acid (PLA), poly(lactic-co-glycolic acid) (PLGA), polyglycolic acid (PGA), and polyhydroxybutyrate (PHB) and mixtures thereof. In addition to PEG, other stabilizer components including proteins, non-ionic hydrophilic polymers, and antibodies can be used to stabilize the noble metal nanoparticles. In some embodiments, mixtures of stabilizing components are useful. All of these suitable stabilizer components must be able to directly covalently bond to the surface of the noble metal nanoparticles.

In the experiments described in this specification, both the stabilizer component and the binding ligand are conjugated onto the surface of the noble metal nanoparticles by gold-thiol binding. In fact, any functional group which exhibits affinity for a noble metal surface, such as a thiol group, an amine group, a phosphine group, a disulfide group, or a mixture thereof could be used for conjugation of the stabilizer component and the binding ligand onto the surface of the noble metal nanoparticles.

In the method of “Sequential Conjugation”, the fabrication of gold nanoparticles bearing a specific number of both PEG 5000 molecules and RGD peptides per gold nanoparticle which will be stable in a biological media of high ionic strength, such as, Dulbecco's Phosphate Buffered Saline (DPBS) or a cell culture medium of Dulbecco's modified Eagle medium (DMEM)) comprises two steps.

Step 1. PEGylated the colloidal gold nanoparticles, in an exemplary embodiment they had an average diameter of 30 nm and were at a concentration of an optical density of OD 1 at their peak LSPR, by mixing them with the SH-PEG 5k solution. The molar ratio of the SH-PEG 5k molecules to the colloidal gold nanoparticles was tuned to be 450 (i.e., PEG/AuNPs=450:1) to keep the stability of the nanoparticles and, at the same time, to avoid excessive surface coverage. FIG. 1B displays the diameter change of 30 nm colloidal gold nanoparticles after being PEGylated with different amount of SH-PEG 5k molecules measured by dynamic light scattering (DLS). As it can be seen from the results shown in FIG. 1B, the minimum molar ratio of thiolated PEG 5k to gold nanoparticles necessary for forming a complete monolayer on the surface of colloidal gold nanoparticles with an average diameter of 30 nm prepared according to the present disclosure is about 1000 and the experimentally adopted molar ratio of 450 is well below it. In this way, enough surface space will be left on the surface of the gold nanoparticles for subsequent RGD peptide conjugation. The mixture was allowed to stand for two hours at room temperature to enable sufficient conjugation of PEG with the gold nanoparticles via thiol-gold bonding.

Step 2. Conjugated the PEGylated gold nanoparticles, average diameter 30 nm, optical density of OD 1, with RGD peptides, SEQ. NO. 1, by mixing it with RGD peptides, the binding ligand in this example. The molar ratio of the RGD peptides to the colloidal gold nanoparticles was selected to be 2000 (i.e., RGD/AuNPs=2000:1) for occupying all surface space left on the surface of the gold nanoparticles after the first step of PEGylation. FIG. 1C displays the hydrodynamic diameters of the partially PEGylated colloidal gold nanoparticles conjugated with different amounts of RGD peptide. The results show the size of the gold nanoparticles increases along with the increase of the molar ratio of RGD peptide molecules to colloidal gold nanoparticles (RGD/AuNP) until it reaches 1600. The experimentally adopted molar ratio of 2000 is well above 1600 and in this way, all surface space left on the surface of the gold nanoparticles after the first step of PEGylation will be occupied by RGD peptides, SEQ. NO. 1. The resultant solutions were allowed to stand for another two hours at room temperature to ensure sufficient conjugation of RGD peptides onto the unoccupied surface space of the gold nanoparticles. The final solutions were centrifuged (5000 g, 10 min in 1.5 ml centrifuge tube) twice with the supernatants removed. The resultant peptide RGD-conjugated 30 nm gold nanoparticles, conjugated nanoparticles, were collected and resuspended to an OD of 10 at their peak LPSR as the stock solution using a buffer solution of 1 mM phosphate buffer (pH 7.4) containing 1 mg/ml bovine serum albumin (BSA).

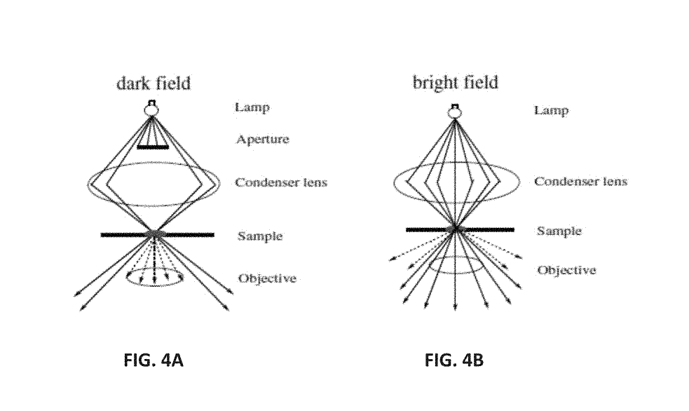

After the fabrication of the peptide RGD-conjugated 30 nm gold nanoparticles, they were used to stain cancer cells for imaging with dark field optical microscopy. FIG. 4A illustrates dark field (left) and FIG. 4B illustrates bright field (right) illumination. Standard bright field illumination relies upon light from the lamp source being gathered by the condenser and shaped into a cone whose apex is focused at the plane of the sample as shown in FIG. 4B. Samples are seen because of their ability to change the speed and the path of the light passing through them. Rather than illuminating the sample with a filled cone of light, dark field illumination is arranged so that the light source is blocked off, causing light to scatter as it hits the sample as shown in FIG. 4A. Dark field illumination is ideal for viewing objects that are transparent, absorb little or no light, or have similar refractive indices as their surroundings, such as small aquatic organisms, oocytes, and cells in tissue culture.

To show the utility of the present disclosure the inventors selected the well-known HeLa cancer cells. Peptide RGD-conjugated gold nanoparticles can specifically target HeLa cells via binding to the integrin receptors overexpressed on HeLa cells through the RGD binding ligand, SEQ. NO. 1. The HeLa cells designated for imaging were cultured in Dulbecco's modification of Eagle's medium (DMEM) plus 10% (v/v) fetal bovine serum (FBS) at 37° C. under 5% CO₂. The cells were first placed in a 35 mm glass-bottomed tissue culture dish and allowed to grow for 2 days. Then a given volume of a stock of RGD-conjugated gold nanoparticles with an optical density of 10 was added to the cell culture dish containing the HeLa cells to achieve a final OD of 1 in the cell culture medium. This represents a concentration of the conjugated nanoparticles of approximately 200 picomolar, sub nanomolar as discussed herein. This level is well below the level of fluorescent marker that would be required to generate a signal. Preferably, all conjugated nanoparticles created according to the present disclosure are used at levels of from 300 picomolar or less to stain cells or tissues. Then, the HeLa cells were incubated for 12 hours at 37° C. and 5% CO₂ in a humidified incubator for the cell to be stained with the gold conjugated nanoparticles. At the end of incubation, the media was gently aspirated from the cell culture dish; the cells were washed with 1 ml Dulbecco's Phosphate Buffered Saline (DPBS), any balanced slat solution could be used, three times to remove free gold conjugates; and the cells were left in the (DPBS). The cells were then ready for imaging by dark field optical microscopy.

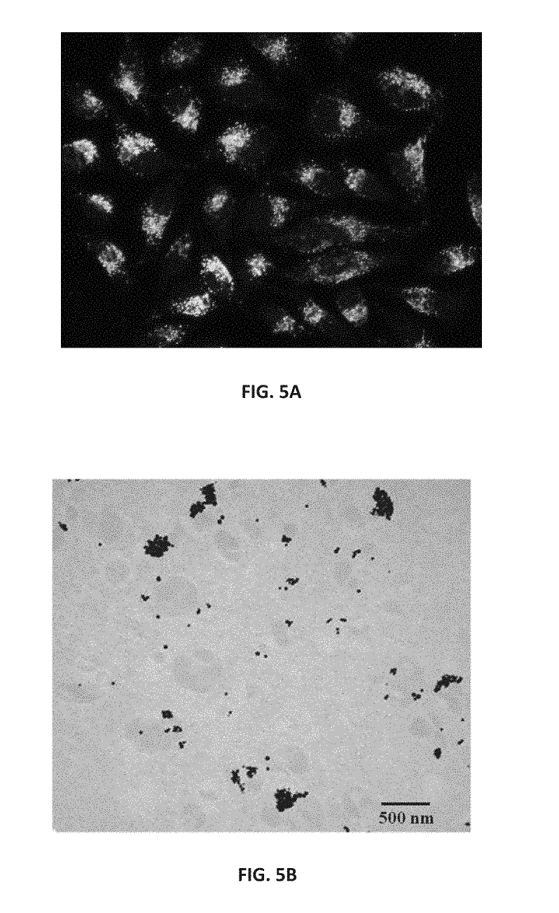

FIG. 5A displays an image of HeLa cancer cells with dark field optical microscopy using the 30 nm colloidal gold conjugated nanoparticles described above as contrast agents. Under dark field illumination, the HeLa cancer cells stained with 30 nm gold nanoparticles appear not green but orange, which is attributed to the formation of gold nanoparticle aggregates in the cytoplasm since the aggregates of 30 nm gold nanoparticles have LSPRs that broaden and shift towards longer wavelengths (known as red-shifting) relative to LSPRs of the individual 30 nm gold nanoparticles, which is around 530 nm. In the image of FIG. 5A the light portions of the cells are the stained portions and the dark portions are unstained. A transmission electron microscopy (TEM) micrograph of intercellular 30 nm gold nanoparticles (black dots) shown in FIG. 5B confirms that the gold nanoparticles exist and aggregated in the cytoplasm. Therefore, under these conditions of incubation it was not possible to predict the color of the cells based on the LSPRs of the individual gold nanoparticles used to stain the cells designated for imaging since the gold conjugated nanoparticles can aggregate after intracellular uptake as demonstrated in the FIG. 5B and aggregates of gold nanoparticles have LSPRs that broaden and shift towards longer wavelengths relative to LSPRs of the individual gold nanoparticles. In order to make cells appear the intrinsic color corresponding to the LSPRs of the individual gold nanoparticles used for cell staining under dark field optical microscopy, it is important to prevent gold nanoparticles from forming aggregates in the cytoplasm. As discussed below, an incubation time of 12 hours as used in these results is too long and permits the conjugated nanoparticles to aggregate.

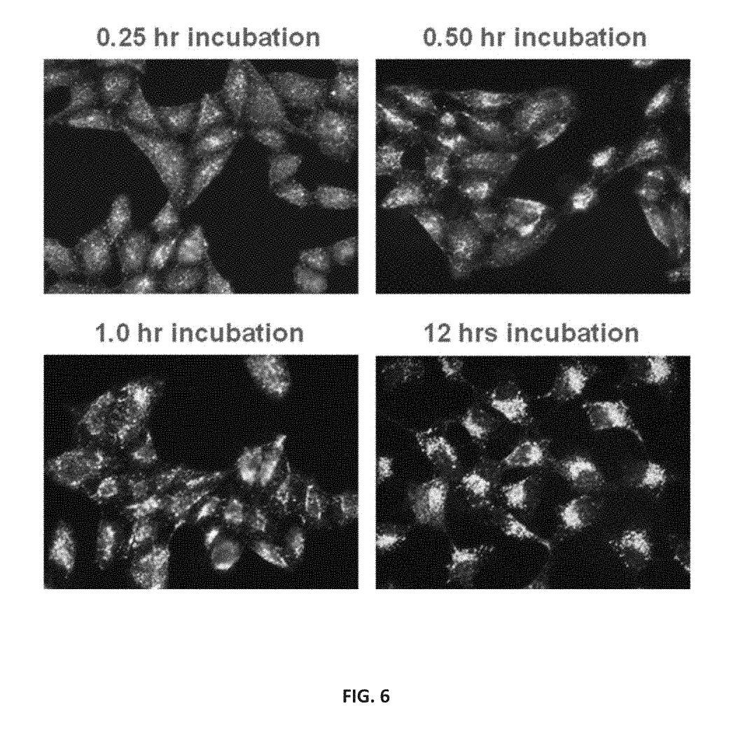

FIG. 6 displays the dependence of the color of HeLa cancer cells stained with 30 nm gold conjugated nanoparticles under dark field optical microscopy on the time period over which the HeLa cancer cells are incubated at 37° C. and 5% CO₂ in a humidified incubator in the presence of peptide RGD-conjugated, SEQ. NO. 1, 30 nm gold nanoparticles, which can specifically target HeLa cells. The HeLa cells were stained as described above with the final optical density (OD) of the gold conjugated nanoparticles in the cell culture medium being 1. It is observed that under dark field illumination, there are changes in the color of the cells stained with 30 nm gold nanoparticles, from mostly appearing green with the incubation time of 0.25 hours to completely appearing orange with the incubation time of 12 hours due to the formation of aggregates of 30 nm gold conjugated nanoparticles in the cytoplasm over the long time period of incubation. Again the light portions of the photographs represent the stained portions and the dark are unstained.

The same phenomena was also observed for HeLa cells stained with 30 nm gold-silver alloy conjugated nanoparticles with 50% gold mole fraction and 50% silver mole fraction (therefore denoted as Au50Ag50) and gold and silver in the each individual nanoparticles are homogeneously mixed. These conjugated nanoparticles were prepared as follows. First, 0.3 nmol of mPEG-SH 5k was added to 1 mL of colloidal Au50Ag50 alloy nanoparticles at an OD of 1. The PEGylation was run for 2 hours at room temperature of 25° C.; then, 1.5 nmol of the RGD peptide, SEQ. NO. 1, was added to the solution and the reaction run for an additional 2 hours. Then the reaction mixture was centrifuged at 5000 g for 10 minutes in a 1.5 ml centrifuge tube, the supernatant was removed to get rid of the unconjugated peptides and for purifying the conjugated nanoparticles. The product was then redispersed to an OD of 10 at its peak LSPR using 1 mM phosphate buffer (pH 7.4) containing 1 mg/ml bovine serum albumin (BSA), the nanoparticle dilution buffer. The same process was used to generate Au80Ag20 conjugated nanoparticles used wherein.

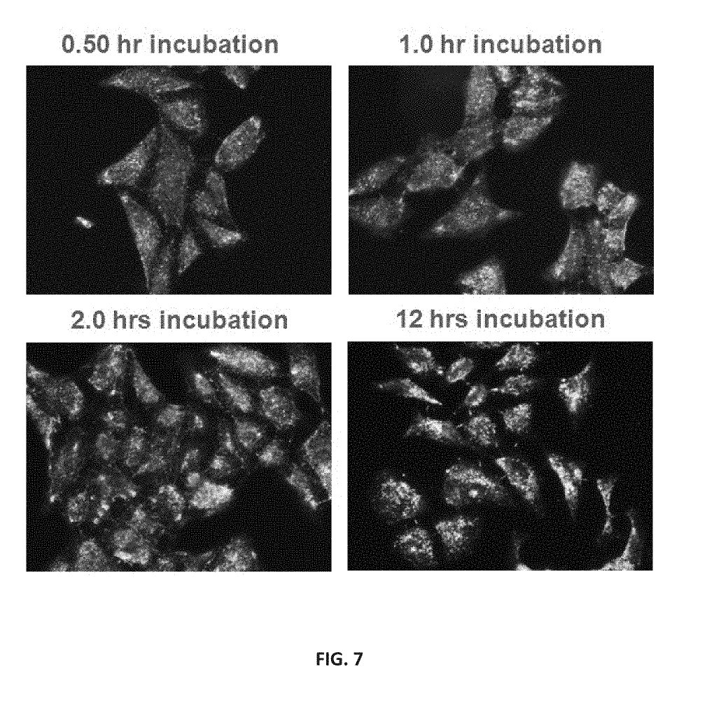

FIG. 7 displays the dependence of the color of the HeLa cells stained with the 30 nm homogeneous Au50Ag50 alloy conjugated nanoparticles under dark field optical microscopy on time period over which HeLa cells are incubated at 37° C. and 5% CO₂ in a humidified incubator in the presence of peptide RGD-conjugated, SEQ. NO. 1, 30 nm Au50Ag50 alloy nanoparticles, which can specifically target HeLa cells, with the final optical density (OD) of Au50Ag50 alloy nanoconjugates in the cell culture medium being 1. It is observed that under dark field illumination, there are changes in the color of cells stained with the 30 nm Au50Ag50 alloy conjugated nanoparticles from mostly appearing blue with the incubation time of 0.5 hr to appearing multiple colors of blue, cyan, and yellow with the incubation time of 12 hrs due to the formation of aggregates of 30 nm Au50Ag50 alloy nanoparticles in the cytoplasm over the long time period of incubation.

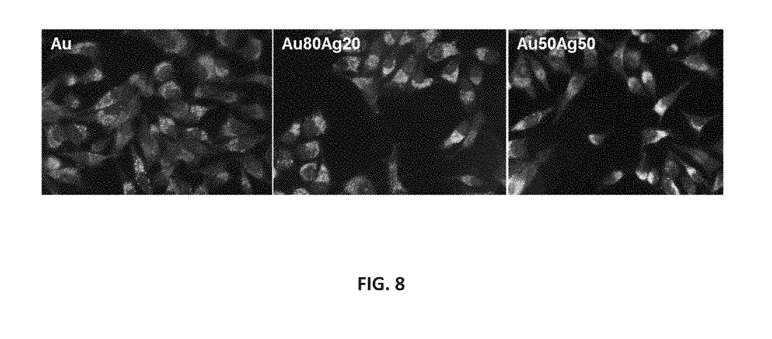

The dependence of the color of HeLa cells stained with either 30 nm gold nanoparticles or 30 nm Au50Ag50 alloy nanoparticles under dark field optical microscopy on time period over which HeLa cancer cells are incubated at 37° C. and 5% CO₂ in a humidified incubator in the presence of gold or Au50Ag50 alloy nanoparticles shown in FIG. 6 and FIG. 7 indicates that the formation of conjugated nanoparticle aggregates in the cytoplasm can be minimized by reducing the time period of incubating the cells designated for imaging in the presence of the conjugated nanoparticles. This is confirmed with the multi-color cell imaging shown in FIG. 8.

The results in the panels of FIG. 8 display multi-color cell imaging with dark field optical microscopy using 30 nm pure gold nanoparticle conjugates (left), 30 nm Au80Ag20 alloy nanoparticle conjugates (middle), and 30 nm Au50Ag50 alloy nanoparticle conjugates (right) as contrast agents. The HeLa cancer cells designated for imaging were stained with 30 nm gold conjugated nanoparticle conjugates (left), 30 nm Au80Ag20 alloy conjugated nanoparticle conjugates (middle), and 30 nm Au50Ag50 alloy conjugated nanoparticle conjugates (right) by incubating them for 2 hours at 37° C. and 5% CO₂ in a humidified incubator in the presence of the respective conjugated nanoparticles added at a final optical density (OD) of gold nanoconjugates in the cell culture medium being 1. It is observed that under dark field illumination, HeLa cancer cells stained with 30 nm gold nanoparticles mostly appear green (left), HeLa cancer cells stained with 30 nm Au80Ag20 alloy nanoparticles mostly appear cyan (middle), and HeLa cancer cells stained with 30 nm Au50Ag50 alloy nanoparticles mostly appear blue. Again the light portions are the stained portions in the black and white images while the dark portions are unstained. These results indicate that with the time of incubating cells designated for imaging at 37° C. and 5% CO₂ in a humidified incubator in the presence of noble metal conjugated nanoparticles being 2 hrs or less, most of the conjugated nanoparticles taken up into the cytoplasm are stable with very few aggregates being formed. Therefore, LSPRs of the noble metal conjugated nanoparticles used for staining cells determine the color of cell imaging under dark field optical microscopy provided the incubation time is kept short enough to prevent aggregation, generally 2 hours or less. As it was discussed above, the LSPRs of the noble metal nanoparticles can be tuned throughout the whole visible region of the electromagnetic spectrum via varying the noble metal nanoparticle compositions, which forms the foundation for the design of a novel method for multi-color cell imaging with dark-field optical microscopy using noble metal nanocolloids as contrast agents as described in the present disclosure.

Examples of binding ligands other than peptides for specifically targeting cells include polymers, deoxyribonucleic acid (DNA) sequences, ribonucleic acid (RNA) sequences, aptamers, amino acid sequences, proteins, peptide-nucleic acid which is an artificially created polymer similar to RNA and DNA, enzymes, antibodies, fluorescent markers, pharmaceutical compounds or mixtures thereof. Using the present process, once the nanoparticles are conjugated to the desired level of stabilizer component the binding ligands can be conjugated to the stabilized nanoparticles either in the original suspension liquid or in a desired biological medium or balanced salt solution. The conjugation is generally carried out by exposure of the stabilized nanoparticles to the binding ligands at a temperature of 25° C. or less for a period of time of at least 1 hour, preferably about 2 hours.

In one embodiment, the present disclosure provides a method for multi-color cell imaging with dark field optical microscopy. In this embodiment, the noble metal conjugated nanoparticles used for cell imaging are peptide-conjugated pure gold nanocolloids, peptide-conjugated Au80Ag20 alloy nanocolloids, or peptide-conjugated Au50Ag50 alloy nanocolloids. In this exemplary embodiment, the cells designated for imaging are the HeLa cells. In a first step the noble metal conjugated nanoparticles were added to the cell culture dish containing the HeLa cells designated for imaging and the cells were incubated in the presence of the noble metal conjugated nanoparticles for less than 2 hours at 37° C. and 5% CO₂ in a humidified incubator to allow for HeLa cell labeling with these noble metal conjugated nanoparticles. In a second step the medium in the cell culture dish was gently aspirated at the end of incubation and then, the cells were washed with Dulbecco's Phosphate Buffered Saline (DPBS), balanced salt solution, three times to remove free noble metal conjugated nanoparticles in solution before imaging. In a third step DPBS buffer was added to the cell culture dish and the HeLa cells were ready for the optical microscopy imaging using dark field illumination with a lamp (halogen or xenon) or light-emitting diode. In a fourth step cell imaging was performed for HeLa cells stained with the noble metal conjugated nanoparticles in the cell culture dish. An inverted optical microscope is preferred for imaging from below the cell culture dish. Limited by the working distance, up to 50× objective lens can be used. Under dark field illumination, cells stained with 30 nm peptide-conjugated pure gold nanocolloids mostly appear green, which is attributed to the exceptional ability of gold nanoparticles to scatter visible light around 530 nm; cells stained with 30 nm peptide-conjugated Au80Ag20 alloy nanocolloids appear mostly appear cyan, which is attributed to the exceptional ability of Au80Ag20 alloy nanoparticles to scatter visible light around 490 nm; and cells stained with 30 nm peptide-conjugated Au50Ag50 alloy nanocolloids mostly appear blue, which is attributed to the exceptional ability of Au50Ag50 alloy nanocolloids to scatter visible light around 450 nm.

In one embodiment, the present disclosure provides a kit executing the method for multi-color cell imaging with dark field optical microscopy described above. This kit comprises (1) peptide-conjugated pure gold nanocolloids, i.e. conjugated nanoparticles, with average size varying between 10 nm to 70 nm; (2) peptide-conjugated Au80Ag20 alloy nanocolloids, i.e. conjugated nanoparticles, with average size varying between 10 nm to 70 nm; (3) peptide-conjugated Au50Ag50 alloy nanocolloids, i.e. conjugated nanoparticles, with average size varying between 10 nm to 70 nm; (4) dilution buffer which is 1 mM phosphate buffer (pH 7.4) containing 1 mg/ml bovine serum albumin; (5) negative control pure gold nanocolloids with no ligand conjugated to them with an average size varying between 10 nm to 70 nm; (6) negative control Au80Ag20 alloy nanocolloids with no ligand conjugated to them and with an average size varying between 10 nm to 70 nm; (7) negative control Au50Ag50 alloy nanocolloids with no ligand conjugated to them and with an average size varying between 10 nm to 70 nm; and (8) dilution containers. The specific binding ligand used is determined by the target cells as discussed herein. The stabilizer component can be any of those described herein so long as it is conjugated at a level of from 30 to 70% of the total surface area of the nanoparticles with rest of the area being taken up by the specific binding ligand.

In the experiments described in this specification, deionized water was selected as the liquid medium. However, other more biological fluids can also be used as the dissolution media. For example, biological fluids can be chosen from, but not limited to: blood, plasma; saliva; urine; buffers such as a phosphate buffer saline (PBS) solution, a buffer for High Performance Capillary Electrophoresis, a hydroxyethyl piperazineethanesulfonic acid (HEPES) sodium salt solution, a citrate-phosphate-dextrose solution, a phosphate buffer solution, a sodium acetate solution, a sodium chloride solution, a sodium DL-lactate solution, a tris(hydroxymethyl) aminomethane ethylenediaminetetraacetic acid (Tris-EDTA) buffer solution, a tris(hydroxymethyl) aminomethane (Tris) buffered saline, or mixtures thereof. For some of the biological fluids, such as serum, one may have to engage in some pre-purification to remove serum proteins which can themselves cause aggregation of the noble metal nanoparticles.

In the experiments described in this specification, noble metal nanoparticles used in the experiments are spherical noble metal nanoparticles with an average diameter of 30 nm. However, colloidal noble metal nanoparticles with other shapes and configurations, including rods, prisms, disks, cubes, core-shell structures, cages, and frames, wherein they have at least one dimension in the range of from 1 to 200 nm, could also work for the colloidal noble metal nanoparticle-based approach developed in the present disclosure for the cell imaging with dark field optical microscopy.

Although the described process of fabrication of colloidal noble metal nanoparticles by laser ablation of bulk noble metal target in a colloidal suspension liquid was illustrated in embodiments wherein the liquid was deionized water, it is possible to carry out the processes described in other liquids. For example, laser ablation of a bulk noble metal target can be carried out in water, methanol, ethanol, acetone, and other organic solvents.

In the experiments described in this specification, the cells designated for imaging were stained with the noble metal conjugated nanoparticles by incubating them at 37° C. and 5% CO₂ in a humidified incubator in the presence of the noble metal conjugated nanoparticles. In principle this temperature could vary between about 10 to 40° Celsius and the CO₂ percentage could vary between 0 to 5% for optimizing the efficiency of the uptake of noble metal conjugated nanoparticles by cells.

In the present disclosure, noble metal conjugated nanoparticles prepared by the method described in this disclosure comprise a noble metal nanoparticle fabricated by a top-down nanofabrication method using bulk noble metal as a source material, at least one stabilizer component, and at least one binding ligand. Both said stabilizer component and said binding ligand contain at least one functional group having an affinity for binding to said noble metal nanoparticle, thereby directly binding both said stabilizer component and said binding ligand onto the surface of said noble metal nanoparticle. Said stabilizer component is present in an amount less than an amount required to provide a 100% monolayer coverage of said stabilizer component on said noble metal nanoparticle. Depending on the identity of the stabilizer components, the identity and ionic strength of the biological buffer, and the identity of the other binding ligands and their levels of use, in most case, said stabilizer component presents in an amount in the range of from 30% to 70% of the number of said stabilizer component equivalent to an amount required to provide a 100% monolayer coverage of said stabilizer component on said noble metal nanoparticle. All unoccupied sites on said noble nanoparticle will be used to conjugate the binding ligand to the same noble metal nanoparticle. Also, the amounts of both said stabilizer component and said binding ligand bound onto surface of said noble metal nanoparticle could be independently adjusted for optimizing both stability and functionality of said noble metal nanoparticle.

Example of an Imaging Kit and Instructions According to the Present Disclosure

### I-colloid™ Gold Nanoparticle Cell Imaging Kit

### 40 nm Gold Nanoparticles

### Instructions for Use

This product is for Research Use Only

### Overview

IMRA's I-colloid™ Gold Nanoparticle Cell Imaging Kit is designed for dark field optical microscopy imaging of cells. The gold nanoparticles are conjugated with a RGD peptide for cell targeting of HeLa cells. Under dark field illumination, gold nanoparticles are 10⁵ to 10⁶ times brighter than organic dyes due to the particles' large optical scattering cross-section at the plasmon resonance wavelength. A low concentration of gold conjugates on the order of nM is sufficient to produce a sharp image. Gold nanoparticles are also resistant to photo-blinking and photo-bleaching, allowing continuous and extended cell imaging, tracking, and analysis. These instructions detail the procedure for cell staining and dark field optical microscopy imaging in cell culture media of HeLa cells using this kit.

### Product Description

Catalog No.: icAu40CI10 (10 Reactions)

Items Included (all Sterilized)

| Item | Material | Description |
|---|---|---|
| No. 1 | Dilution Buffer | 10x concentrated, 2 ml |
| No. 2 | Dilution container | Micro tube, 2 ml, Polypropylene (Sarstedt, cat. |
| | | no. 72.694.106) |
| No. 3 | Gold conjugates | 40 nm gold nanoparticle RGD peptide |
| | | conjugates, 1 ml, OD 10 (0.5 mg/ml) |
| No. 4 | Negative control | 40 nm gold nanoparticles, 0.5 ml, OD 10 |
| | | (0.5 mg/ml) |

### Safety Precautions

Standard safety precautions in handling laboratory reagents should be adhered to.

### Product Compatibility

The gold conjugates are stable in the solutions as provided. High ionic strength (e.g., >0.25 M NaCl) reagents will destabilize the colloid and induce aggregation. Dilution can be made by the dilution buffer (item No. 1) or a cell culture media during cell staining (see step II below).

### Additional Materials and Equipment Needed

Cancer cells of interest (e.g., human HeLa cells) cultured aseptically at 37° C. and 5% CO₂ in a humidified incubator
Imaging dish: 35 mm glass bottom cell culture dishes (MatTek Corporation, Part No. P35G-0-14-C)
Cell culture media: Dulbecco's modified Eagle medium (DMEM, Thermo Fisher Scientific, cat. no. 11995-065) supplemented with 10% (v/v) % fetal bovine serum (FBS) and 1% penicillin-streptomycin (100 I.U./ml of penicillin and 100μ/ml streptomycin).
Cell rinse buffer: Dulbecco's Phosphate Buffered Saline (DPBS, Thermo Fisher Scientific, cat. no. 14190-144)
Optical microscope: with dark field illumination. Inverted scope is preferred for imaging from below the imaging dish.
Standard biological laboratory and cell culture equipment such as pipettes, class II biological safety cabinet, and CO₂ incubator for growing and maintaining cell cultures.

### Cell Staining and Imaging Procedure

The procedure below is based on staining and imaging human HeLa cancer cells. Protocols should be modified to meet the individual application.

I. Cell Preparation
1. Transfer 1 ml suspension of cells of interest at a density between 1×10⁴ cells/ml to 2×10⁴ cells/ml prepared in cell culture medium into a new imaging dish and culture for an additional 24 hours at 37° C. and 5% CO₂ in a humidified incubator to allow the cells to attach to surface of the imaging dish prior to initiating staining with the provided 40 nm gold conjugates.

II. Gold Conjugate Dilution
1. Bring all items of the cell imaging kit and cell culture reagents into the biological safety cabinet. Allow the items and reagents to come to room temperature.
2. Use a pipette to mix the gold conjugates (item No. 3) by pipetting up and down for a few times and then transfer 100 μl of the conjugates solution to the dilution container (item No. 2). The dilution container can be reused later on after rinsing with cell rinse buffer for three times.
3. Dilute 100 μl of gold nanoparticle conjugate solution from OD 10 (0.5 mg/ml) to OD 1 (0.05 mg/ml) by adding 900 μl cell culture media and mixing by pipetting up and down several times.

III. Cell Staining
1. Aspirate the original cell culture media from the imaging dish. Wash cells twice with 1 ml of cell rinse buffer.
2. Add 1 ml of gold conjugate solution of OD 1 (0.05 mg/ml) as prepared in step 11.3 to the cell imaging dish. Incubate for 1 hour at 37° C. and 5% CO₂ in a humidified incubator for the cell to be stained with the gold nanoparticles.
3. At the end of the incubation time, gently aspirate the media from the imaging dish. Wash the cells with 1 ml cell rinse buffer three times to remove free gold conjugates. Leave the cells in the rinse buffer. The cells are ready for optical microscopy imaging.

IV. Imaging
1. An inverted optical microscope is preferred for imaging from below the imaging dish. Limited by the working distance, up to ×50 objective lens can be used. Under dark field illumination, cells stained with pure gold nanoparticles mostly appear green, which is attributed to enhanced light scattering by the gold nanoparticles. Yellow and red color occasionally appear at high concentration.
2. It is recommend to run a negative control test to confirm the specification of cell staining by replacing the gold conjugates with the negative control (item No. 4) in steps II and III.

V. Cell Fixation (OPTIONAL)
Cell fixation is suggested if long-term cell imaging and analysis is required.
After cell staining, the cells can be fixed onto the imaging dish by adding 0.1 ml fresh 4% paraformaldehyde in PBS and incubation at ambient conditions for 15 minutes followed by washing three times with 1 ml of cell rinse buffer. Leave the cells in the cell rinse buffer. The cells are ready for the optical microscopy imaging. Store the fixed cells at 4° C. when they are not being used. Do not freeze.

### Release Information

The functionality of specific cell staining of the gold conjugates for optical microscopy imaging under dark field illumination has been confirmed with human HeLa cells and compared with negative control using non-functionalized gold nanoparticles.

### Shipping and Storage

This product is shipped in ambient conditions. Store at 2° C.-8° C. upon receiving the product. Remaining materials after use should be retained in the supplied container and sealed for future use. Do not expose to temperatures above 60° C. Do not freeze.

### Technical Support

For questions regarding this product and technical support please visit our website http://nano.imra.com or contact us via telephone or email.

Thus, while only certain embodiments have been specifically described herein, it will be apparent that numerous modifications may be made thereto without departing from the spirit and scope of the disclosure. Further, acronyms are used merely to enhance the readability of the specification and claims. It should be noted that these acronyms are not intended to lessen the generality of the terms used and they should not be construed to restrict the scope of the claims to the embodiments described therein. It is intended that the disclosure be limited only by the claims which follow, and not by the specific embodiments and their variations and combinations as described herein-above.

What is claimed is:

1. A method for multi-color cell imaging with dark field optical microscopy comprising the following steps:
a) adding a plurality of noble metal conjugated nanoparticles to a cell culture dish containing cell culture medium and a plurality of cells designated for imaging and incubating them together for a period of time of less than 2 hours;
b) after incubation, aspirating the cell culture medium from the cell culture dish and washing the cells with a rinse buffer comprising a balanced salt solution to remove free noble metal conjugated nanoparticles from the dish;
c) adding rinse buffer to the cell culture dish containing the washed cells; and
d) performing cell imaging of the cells with an optical microscope using dark field illumination wherein the color of the labeled cells is determined by the localized surface plasmon resonances of the individual noble metal conjugated nanoparticles.

2. The method as recited in claim 1, wherein step a) comprises providing a plurality of conjugated noble metal nanoparticles comprising noble metal nanoparticles having from 30 to 70% of their surface covered by a bound stabilizer component, the stabilizer component keeping the nanoparticles stable in a biological fluid, a balanced salt solution and a cell cytoplasm, and the remainder of the surface of the nanoparticles covered by a binding ligand that specifically binds to the cells designated for imaging.

3. The method as recited in claim 2, comprising providing a plurality of noble metal nanoparticles having at least one dimension in the range of from 1 to 200 nanometers.

4. The method as recited in claim 2, comprising providing a plurality of noble metal nanoparticles having a shape of selected from the group consisting of a sphere, a rod, a prism, a disk, a cube, a core-shell structure, a cage, a frame, or a mixture thereof.

5. The method as recited in claim 2, comprising providing a plurality of noble metal nanoparticles having a composition selected from the group consisting of gold, silver, copper, or a mixture thereof.

6. The method as recited in claim 2, comprising providing a stabilizer component selected from the group consisting of a polyethylene glycol (PEG), a protein, a non-ionic hydrophilic polymer, an antibody, or a mixture thereof.

7. The method as recited in claim 2, comprising providing a binding ligand selected from the group consisting of a deoxyribonucleic acid (DNA) sequence, a ribonucleic acid (RNA) sequence, an aptamer, a peptide, an antibody, a peptide-nucleic acid, or mixtures thereof.

8. The method as recited in claim 1, comprising providing as the cells designated for imaging cancer cells.

9. The method as recited in claim 1, comprising providing as the cell culture medium Dulbecco's modified Eagle medium supplemented with 10% (v/v) fetal bovine serum and optionally 1% penicillin-streptomycin (100 I.U./ml penicillin and 100 μg/ml streptomycin).

10. The method as recited in claim 1, wherein the rinse buffer is Dulbecco's Phosphate Buffered Saline.

11. A kit executing the method for multi-color cell imaging as described in claim 1 comprising: a) a plurality of conjugated noble metal conjugated nanoparticles; b) a plurality of negative control noble metal nanoparticles; c) a dilution buffer; d) a plurality of dilution containers; and e) instructions for use of said kit, wherein said instructions describe: cell preparation, noble metal conjugated nanoparticle dilution, cell staining, optional cell fixation, and imaging of cells.

12. The kit as recited in claim 11, wherein each of said plurality of conjugated noble metal nanoparticles comprise a noble metal nanoparticle having from 30 to 70% of its surface covered by a stabilizer component and a binding ligand covering the remainder of said surface, said stabilizer component keeping said conjugated noble metal nanoparticle stable in a biological fluid, a balanced salt solution and a cell cytoplasm and said binding ligand specifically binding to cells designated for imaging.

13. The kit as recited in claim 12, wherein said plurality of noble metal nanoparticles and said plurality of negative control noble metal nanoparticles each have at least one dimension in the range of from 1 to 200 nanometers.

14. The kit as recited in claim 12, wherein said plurality of noble metal nanoparticles and said plurality of negative control noble metal nanoparticles each have a shape selected from the group consisting of a sphere, a rod, a prism, a disk, a cube, a core-shell structure, a cage, a frame, or a mixture thereof.

15. The kit as recited in claim 12, wherein said plurality of noble metal nanoparticles and said plurality of negative control noble metal nanoparticles have a composition selected from the group consisting of gold, silver, copper, or a mixture thereof.

16. The kit as recited in claim 12, wherein said stabilizer component is selected from the group consisting of polyethylene glycol (PEG), a protein, a non-ionic hydrophilic polymer, an antibody, or a mixture thereof.

17. The kit as recited in claim 12, wherein said binding ligand is selected from the group consisting of a deoxyribonucleic acid (DNA) sequence, a ribonucleic acid (RNA) sequence, an aptamer, a peptide, an antibody, a peptide-nucleic acid, or mixtures thereof.

18. The kit as recited in claim 11, wherein said negative control noble metal nanoparticle comprises a noble metal nanoparticle and a stabilizer component, said stabilizer component keeping said noble metal nanoparticle stable in a biological fluid, a balanced salt solution and a cell cytoplasm.

19. The kit as recited in claim 11, wherein said dilution buffer comprises 1 mM phosphate buffer, pH 7.4, containing 1 mg/ml bovine serum albumin (BSA).

20. The kit as recited in claim 11, wherein the cells designated for imaging are cancer cells.

21. The kit as recited in claim 11, wherein said dilution containers are sterile 2 ml polypropylene microtubes.
